(19) United States
(12) Patent Application Publication
(10) Pub. No.: US 2022/0015824 A1
(43) Pub. Date: Jan. 20, 2022

(54) TREATMENT TOOL

(71) Applicant: OLYMPUS CORPORATION, Tokyo (JP)

(72) Inventor: Satoshi Horie, Tokyo (JP)

(73) Assignee: OLYMPUS CORPORATION, Tokyo (JP)

(21) Appl. No.: 17/491,628

(22) Filed: Oct. 1, 2021

Related U.S. Application Data
(63) Continuation of application No. PCT/JP2019/014873, filed on Apr. 3, 2019.

(51) Int. Cl. A61B 18/14

(52) U.S. Cl. CPC A61B 18/1485; A61B 2018/00184

(57) ABSTRACT

A treatment tool includes: an insertion portion including a plurality of holes; a shaft that is arranged in the insertion portion; a plurality of needles that are connected to the shaft, each needle being configured to move between a first position and a second position; a plurality of first insulating materials, each insulating material being configured to cover the needle; and an energy supplier configured to supply electric power to the plurality of needles. The hole is configured to regulate a protruding direction of the needle to a direction intersecting with the longitudinal direction of the insertion portion. When an area that is positioned outside the insertion portion out of a whole area of the needle at the first position is sectioned into two areas that are a first area and a second area, the first insulating material is configured to cover the second area.

### CROSS-REFERENCE TO RELATED APPLICATION

This application is a continuation of International Application No. PCT/JP2019/014873, filed on Apr. 3, 2019, the entire contents of which are incorporated herein by reference.

### BACKGROUND

### 1. Technical Field

The disclosure relates to a treatment tool.

### 2. Related Art

A treatment tool that treats a region serving as a subject to be treated (“subject region” below) in living tissue by applying energy to the subject region has been known (for example, Japanese Laid-open Patent Publication No. 2002-28166).

The treatment tool described in Japanese Laid-open Patent Publication No. 2002-28166 is a nasal treatment tool that inserts two needles (needle electrodes) into the inferior turbinate and flows a high frequency current from the two needles to the inferior turbinate. The treatment tool includes an insertion portion that is cylindrical and elongated and the two needles that are arranged in the insertion portion and that move forward and backward along the longitudinal direction of the insertion portion.

### SUMMARY

In some embodiments, a treatment tool includes: an insertion portion that is hollow and elongated, the insertion portion including a plurality of holes, each hole being configured to communicate between an inside and an outside of the insertion portion; a shaft that is arranged in the insertion portion, the shaft being configured to move forward and backward in a longitudinal direction of the insertion portion; a plurality of needles that are connected to the shaft, each needle being configured to move between a first position and a second position according to forward and backward move of the shaft, the first positon being a position in which the needle protrudes from the hole to the outside of the insertion portion, the second position being a positon in which the needle is positioned in the insertion portion; a plurality of first insulating materials, each insulating material being configured to cover the needle; and an energy supplier configured to supply electric power to the plurality of needles. The hole is configured to regulate a protruding direction of the needle to a direction intersecting with the longitudinal direction of the insertion portion, the protruding direction of the needle being a direction in which the needle protrudes to the outside of the insertion portion, and when an area that is positioned outside the insertion portion out of a whole area of the needle at the first position is sectioned into two areas that are a first area and a second area, the first insulating material is configured to cover the second area, the first area being an area containing a distal end of the needle, the second area being an area excluding the first area.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to a first embodiment;

FIG. 2 is a diagram illustrating a distal end of an insertion portion;

FIG. 3 is a diagram illustrating the distal end of the insertion portion;

FIG. 4 is a diagram illustrating a base;

FIG. 5 is a diagram illustrating distal ends of a plurality of needles;

FIG. 6 is a diagram illustrating a situation of an operation using the treatment system;

FIG. 7 is a diagram illustrating distal ends of a plurality of needles according to a second embodiment;

FIG. 8 is a diagram illustrating a modification of the first and second embodiments; and

FIG. 9 is a diagram illustrating the modification of the first and second embodiments.

### DETAILED DESCRIPTION

With reference to the accompanying drawings, modes for carrying out the disclosure (“embodiments” below) will be described below. The embodiments to be described below do not limit the disclosure. In the illustration of the drawings, the same components are denoted with the same reference numerals.

### First Embodiment

### Schematic Configuration of Treatment System

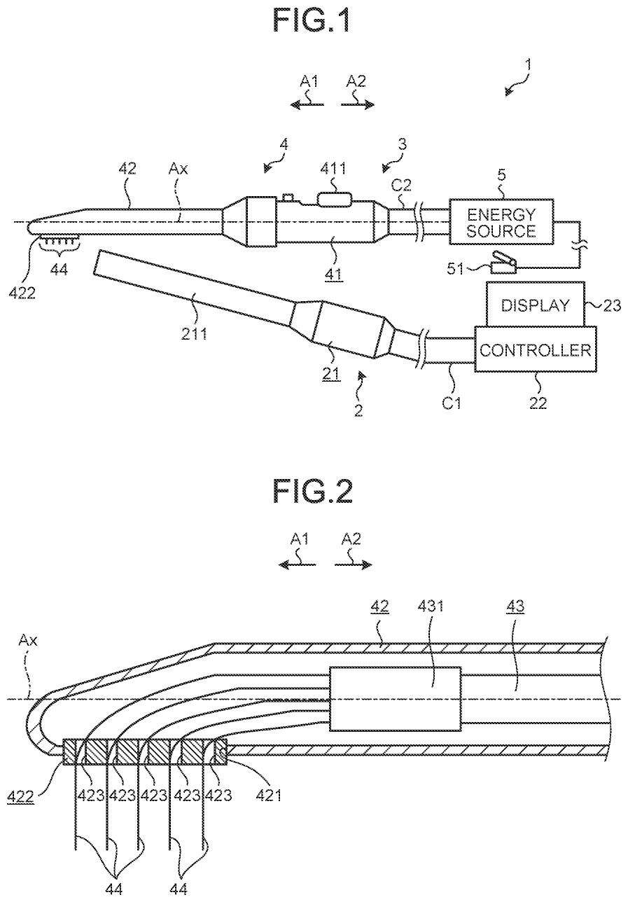

A treatment system 1 treats a region serving as a subject to be treated (“subject region” below) in living tissue by applying energy to the subject region. Treating a subject region means cauterizing part of a mucosa layer and posterior nasal nerves in the inferior turbinate. As illustrated in FIG. 1, the treatment system 1 includes an endoscope device 2 and a treatment tool 3.

As illustrated in FIG. 1, the endoscope device 2 includes an endoscope 21, a controller 22, and a display 23.

The endoscope 21 includes an endoscope insertion portion 211 (FIG. 1) that is elongated, and the endoscope 21 loads a subject image from a distal end of the endoscope insertion portion 211 and captures the subject image. The endoscope insertion portion 211 may be formed into a hard state such that the shape of the endoscope insertion portion 211 is maintained or may be formed into a flexible state such that the endoscope insertion portion 211 can be curved as appropriate.

The controller 22 is electrically connected to the endoscope 21 via an electric cable C1 and controls operations of the endoscope 21. The controller 22 controls operations of the display 23 and causes the display 23 to display the image that is captured by the endoscope 21.

The display 23 consists of a display using liquid crystals, organic electro luminescence (EL), or the like, and displays the image that is captured by the endoscope 21.

The treatment system 1 does not necessarily need the endoscope device 2.

The treatment tool 3 inserts a plurality of needles into the inferior turbinate and flows a high frequency current from the needles to the inside of the inferior nasal conch, thereby cauterizing part of posterior nasal nerves in the inferior turbinate.

A detailed configuration of the treatment tool 3 will be described below.

### Configuration of Treatment Tool

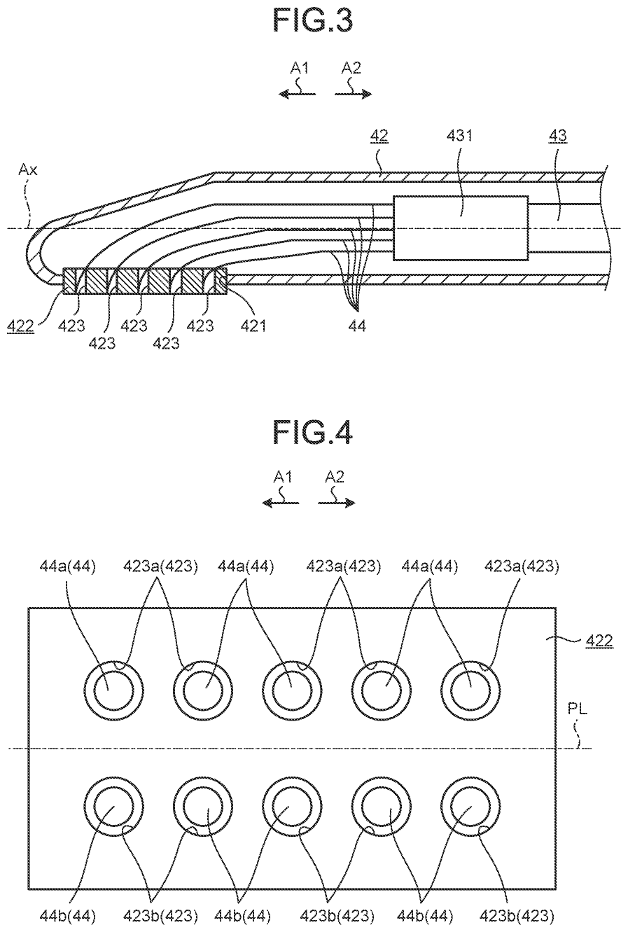

As illustrated in FIG. 1, the treatment tool 3 includes a treatment tool body 4 including a handle 41, a treatment tool insertion portion 42, a shaft 43 (refer to FIG. 2 and FIG. 3), and a plurality of needles 44; and an energy source 5 that is electrically connected to the treatment tool body 4 via the electric cable C2.

One side along a center axis Ax (FIG. 1) of the treatment tool insertion portion 42 is referred to as a distal end side A1 (FIG. 1) and the other side is referred to as a proximal end side A2 (FIG. 1) below.

The handle 41 is a part that is held by an operator by hand and the handle 41 extends along the center axis Ax and has a shape of a cylinder that has a bottom and that is open on the distal end side A1. As illustrated in FIG. 1, an operation member 411 that is movable to the distal end side A1 or the proximal end side A2 according to an operation by the operator is arranged in a state of being exposed to the outside of the handle 41.

The treatment tool insertion portion 42 corresponds to an insertion portion. The treatment tool insertion portion 42 consists of a hollow and elongated member. More specifically, the treatment tool insertion portion 42 extends along the center axis Ax and has a shape of a cylinder that has a bottom and that is open on the proximal end side A2. An end of the treatment tool insertion portion 42 on the proximal end side has a diameter larger than other parts. The treatment tool insertion portion 42 is attached to the handle 41 with an end of the handle 41 on the distal end side A1 being inserted into the treatment tool insertion portion 42 from the proximal end side A2. The treatment tool insertion portion 42 is attached to the handle 41 rotatably in a circumferential direction about the center axis Ax.

FIG. 2 and FIG. 3 are diagrams illustrating a distal end of the treatment tool insertion portion 42. Specifically, FIG. 2 and FIG. 3 are cross-sectional views of the distal end of the treatment tool insertion portion 42, taken along a plane parallel to the surface of FIG. 1.

As illustrated in FIG. 2 or FIG. 3, through-holes 421 penetrating the distal end of the treatment tool insertion portion 42 from the inside to the outside of the treatment tool insertion portion 42 are formed in the distal end. The through-holes 421 are sealed with a base 422.

FIG. 4 is a diagram illustrating the base 422. Specifically, FIG. 4 is a diagram of the base 422 in FIG. 2 and FIG. 3 viewed from the bottom side. Note that PL presented on FIG. 4 denotes a plane that includes the center axis Ax and that is parallel to the surfaces of FIG. 2 and FIG. 3.

The base 422 is a flat board that is formed of an electrical insulating material and that has front and back surfaces each of which is orthogonal to the plane PL. A plurality of holes 423 penetrating the base 422 from the front surface to the back surface are formed in the base 422.

Each of the holes 423 extends in a direction intersecting with the center axis Ax (the longitudinal direction of the treatment tool insertion portion 42). In the first embodiment, in a state of being parallel with each other, the holes 423 extends in the direction intersecting with the center axis Ax. Ten holes 423 are formed. Separating from the plane PL by a given distance, five holes 423 out of the ten holes (first holes 423a (FIG. 4) below) are arranged in parallel. The remaining five holes 423 (second holes 423b (FIG. 4) below) are arranged in positions such that the second holes 423b and the first holes 423a are symmetric with respect to the plane PL.

The shaft 43 is a member having a shape of a cylinder that extends from the inside of the handle 41 to the treatment tool insertion portion 42. More specifically, in a state of being allowed to move forward and backward along the center axis Ax and being regulated in rotating circumferentially about the center axis Ax, the shaft 43 is attached to the treatment tool insertion portion 42. In other words, the shaft 43 is rotatable with the treatment tool insertion portion 42 circumferentially about the center axis Ax. Although specific illustration is omitted, the end of the shaft 43 on the proximal end side A2 is joined to the operation member 411. More specifically, in the state of being allowed to rotate circumferentially about the center axis Ax and being regulated in moving forward and backward along the center axis Ax, the end of the shaft 43 on the proximal end side A2 is attached to the operation member 411. In the words, the shaft 43 is movable forward and backward along the center axis Ax together with the operation member 411 (FIG. 2 and FIG. 3).

As illustrated in FIG. 2 or FIG. 3, a fixation portion 431 for fixing the needles 44 is arranged at the distal end of the shaft 43.

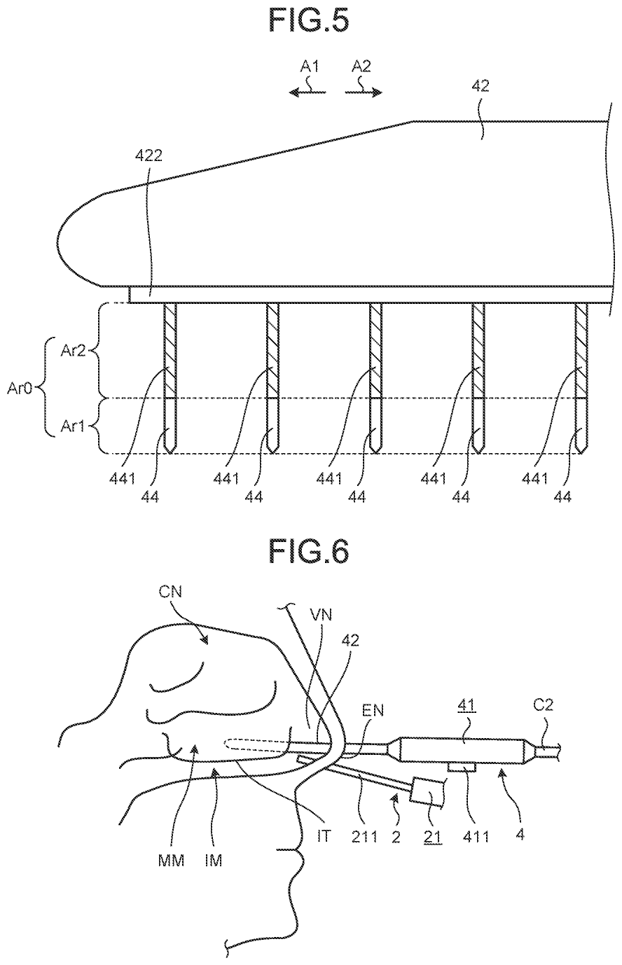

The same number of (ten in the first embodiment) the needles 44 as that of the holes 423 are arranged. Each of the proximal ends of the needles 44 is fixed by the fixation portion 431 and, according to forward and backward move of the shaft 43, the needles 44 move between first positions (FIG. 2) in which the needles 44 protrude from the holes 423, respectively, to the outside of the treatment tool insertion portion 42 and second positions (FIG. 3) in which the respective needles 44 are positioned in the treatment tool insertion portion 42. Each needle 44 is partly covered with a first insulating material 441 (see FIG. 5). The first insulating material 441 is formed of an electrically insulating material.

FIG. 5 is a diagram illustrating the distal ends of the needles 44. Specifically, FIG. 5 is a diagram of the needles 44 in a state of being positioned in the first positions, viewed from a direction orthogonal to the plane PL.

For convenience of description, an area Ar0 that is positioned outside the treatment tool insertion portion 42 out of the whole area of the needle 44 at the first position is sectioned into two areas that are a first area Ar1 and a second area Ar2 below. The first area Ar1 is an area containing the tip of the needle 44. The second area Ar2 is an area excluding the first area Ar1.

The first insulating material 441 covers the second area Ar2 of the needle 44.

In the first embodiment, the first area Ar1 that is not covered with the first insulating material 441 corresponds to an energy emission area. For convenience of description, the first area Ar1 is referred to as an energy emission area Ar1 below.

All the lengths of the respective second areas Ar2 of the needles 44 (lengths in the direction in which the needles 44 extend) are set equal to one another. It is preferable that the lengths of the second areas Ar2 be 1 mm or larger. All the lengths of the respective energy emission areas Ar1 of the needles 44 (lengths in the direction in which the needles 44 extend) are set equal to one another.

In the first embodiment, the needles 44 consists of superelastic wires (Ni—Ti) to which no special processing (bending) is added. In other words, because no special processing (such as curing) is added to the needles 44, when the needles 44 move to the first positions (FIG. 2 and FIG. 5), the directions in which the needles 44 protrude to the outside of the treatment tool insertion portion 42 are regulated by the holes 423 and the needles 44 protrude to the outside of the treatment tool insertion portion 42 along the direction in which the holes 423 extend (the direction orthogonal to the center axis Ax).

The needles 44 are not limited to superelastic wires and SUS (WPB members) formed into springs may be used.

Each of the proximal ends of the needles 44 is electrically connected through the shaft 43 to the electric cable C2 that is laid to the end of the shaft 43 on the distal end side A1.

For convenience of description, the needles 44 that are inserted into the first holes 423a out of the needles 44 are referred to as first needles 44a (FIG. 4) and the needles 44 that are inserted into the second holes 423b are referred to as second needles 44b (FIG. 4).

The energy source 5 supplies high frequency power between the five first needles 44a and the five second needles 44b via the electric cable C2 in response to an operation of the operator on a switch 51, such as a foot switch (FIG. 1). In other words, the energy source 5 corresponds to an energy supplier.

### Circumstances of Operation Using Treatment System

Circumstances of an operation using the treatment system 1 will be described next.

FIG. 6 is a diagram illustrating circumstances of the operation using the treatment system 1.

First of all, as illustrated in FIG. 6, the operator inserts the distal end of the endoscope insertion portion 211 into a cavity of nose CN through an external naris EN of a patient. The operator operates the operation member 411 to position the needles 44 in the second positions. While checking the captured image (image of the inside of the cavity of nose CN) that is captured by the endoscope 21 and that is displayed on the display 23, the operator inserts the distal end of the treatment tool insertion portion 42 toward the inferior turbinate IT in the cavity of nose CU via the external naris EN, a vestibulum nasi VN, and, for example, an inferior meatus IM. The operator pushes the outer surface of the base 422 against tissue surface of the inferior turbinate IT.

The operator then operates the operation member 411 to position the needles 44 in the first positions. Accordingly, the needles 44 are inserted into the inferior turbinate IT. The dimension of length of the parts of the needles 4 (areas Ar0) that are inserted into the inferior turbinate IT differs per subject tissue and is around 1 to 5 mm.

The operator then operates the switch 51. Thus, high frequency power is supplied between the first needles 44a and the second needles 44b from the energy source 5. Accordingly, a high frequency current flows through part of the mucosa layer and posterior nasal nerves (not illustrated in the drawings) are positioned between the energy emission areas Ar1 of the first needles 44 and the energy emission areas Ar1 of the second needles 44b. In other words, high frequency energy is supplied to part of the posterior nasal nerves. Accordingly, the posterior nasal nerves are partly cauterized.

According to the first embodiment, the following effect is made.

In the treatment tool 3 according to the first embodiment described above, the directions in which the needles 44 protrude to the outside of the treatment tool insertion portion 42 are regulated by the holes 423 to the direction intersecting with the center axis Ax. Thus, when treating a subject region, such as the inferior turbinate IT, that is positioned in the direction intersecting with the direction of insertion of the treatment tool insertion portion 42 into the cavity of nose CN, it is not necessary to largely slope the treatment tool body 4 with respect to the direction of insertion. Thus, according to the treatment tool 3 according to the first embodiment, it is possible to increase convenience.

Particularly, because the configuration in which the holes 423 regulate the directions of protrusion of the needles 44 to the outside of the treatment tool insertion portion 42 is employed, it is not necessary to additionally arrange a mechanism that regulates the directions of protrusion of the needles 44. Thus, it is possible to simplify the configuration and reduce the diameter of the treatment tool insertion portion 42.

When application of a high frequency energy to the tissue surface of the inferior turbinate IT cauterizes the tissue surface, there is not only a problem in that uncomfortableness, such as pain, is given to the patient but also a problem in that recovery delays and there is a great effect on the functions of tissue and internal organs.

In the first embodiment, the second areas Ar2 of the needles 44 are covered with the first insulating materials 441, respectively. Thus, a high frequency current flows only between the energy emission areas Ar1 of the needles 44 on the distal end side. In other words, the cauterized area does not reach the tissue surface of the inferior turbinate IT, which makes it possible to avoid the above-described problems.

Particularly, all the lengths of the respective second areas Ar2 of the needles 44 (the lengths in the direction in which the needles 44 extend) are set equal to one another. It is preferable that the length of the second area Ar2 be 1 mm or larger. All the lengths of the energy emission areas Ar1 of The needles 44 (the lengths in the direction in which the needles 44 extend) are set equal to one another. In other words, all the positions of protrusion of the energy emission areas Ar1 of the needles 44 from the base 422 are set the same. Accordingly, it is possible to stably treat a certain region inside by a given dimension from the tissue surface of the inferior turbinate IT.

### Second Embodiment

A second embodiment will be described next.

In the following description, the same components as those of the above-described first embodiment are denoted with the same reference numerals and detailed description thereof will be omitted or simplified.

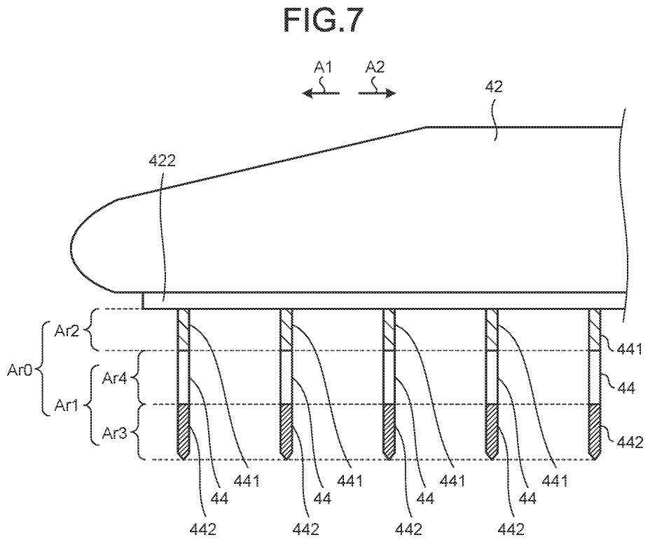

FIG. 7 is a diagram illustrating distal ends of the needles 44 according to the second embodiment. Specifically, FIG. 7 is a diagram corresponding to FIG. 5 and is a diagram of the needles 44 being positioned in the first positions, viewed from a direction perpendicular to a plane PL.

In the second embodiment, as illustrated in FIG. 7, the needles 44 are covered with second insulating materials 442 in addition to the first insulating materials 441. Like the first insulating materials 441, the second insulating materials 442 are formed of an electrical insulating material.

For convenience of description, as in the above-described first embodiment, an area Ar0 that is positioned outside of the treatment tool insertion portion 42 out of the whole area of the needle 44 in the state of being positioned in the first position is sectioned into two areas that are a first area Ar1 and a second area Ar2 below. The first area Ar1 is sectioned into two areas that are a third area Ar3 and a fourth area Ar4. The third area Ar3 is an area containing the distal end of the needle 44. The fourth area Ar4 is an area excluding the third area Ar3.

Like the above-described first embodiment, the first insulating materials 441 cover the second areas Ar2 of the needles 44. On the other hand, the second insulating materials 442 cover the third areas Ar3. In other words, a fourth area Ar4 that is not covered with the first and second insulating materials 441 and 442 corresponds to the energy emission area. For convenience of description, the fourth area Ar4 is referred to as an energy emission area Ar4 below.

All the lengths of the respective second areas Ar2 of the needles 44 (lengths in the direction in which the needles 44 extend) are set equal to one another. It is preferable that the length of the second areas Ar2 be 1 mm or larger. All the lengths of the respective energy emission areas Ar4 of the needles 44 (lengths in the direction in which the needles 44 extend) are set equal to one another.

According to the above-described second embodiment, the following effect is made in addition to the same effect as that of the above-described first embodiment.

The second embodiment realizes a structure that is optimum to define the lengths of the needles 44 that are embedded in living tissue by pushing the distal ends of the needles 44 against hard tissue, such as a bone or cartilage. In other words, because the third areas Ar3 of the needles 44 are covered with the second insulating materials 442, high-frequency energy is not applied to hard tissue, such as a bone or cartilage, against which the distal ends of the needles 44 are pushed, which enables application of high frequency energy to only desired tissue.

### Other Embodiments

The modes for carrying out the disclosure has been described but the disclosure should not be limited only by the first and second embodiments described above.

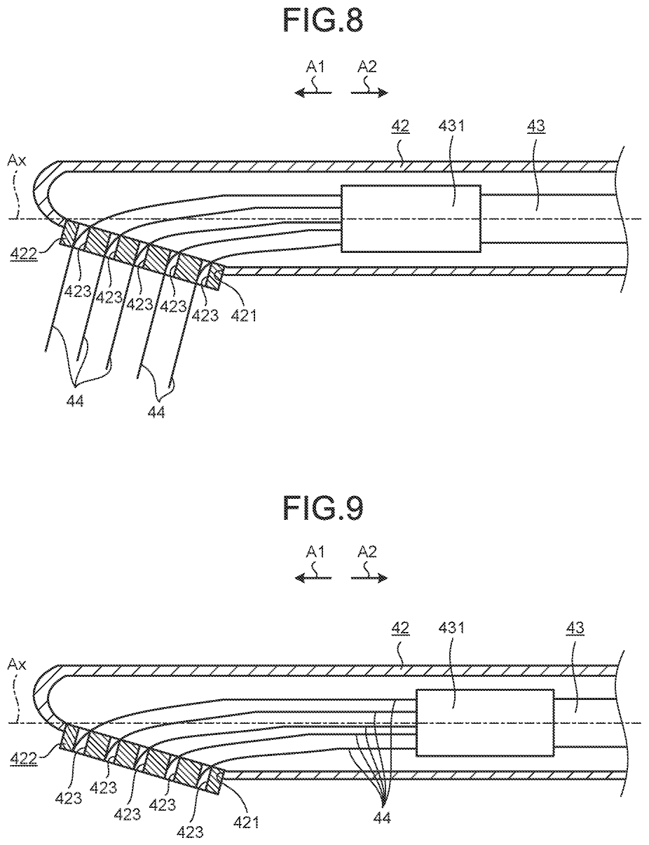

FIGS. 8 and 9 are diagrams illustrating a modification of the first and second embodiments. Specifically, FIG. 8 is a cross-sectional view that corresponds to FIG. 2 and illustrates that the needles 44 are positioned in the first positions. FIG. 9 a cross-sectional view that corresponds to FIG. 3 and illustrates that the needles 44 are positioned in the second positions.

In the above-described first and second embodiments, the needles 44 protrude in the direction orthogonal to the center axis Ax; however, the needles 44 are not limited thereto. For example, a configuration in which, as illustrated in FIG. 8 and FIG. 9, the base 422 is formed in a posture such that the direction in which the holes 423 extend intersects with the center axis Ax and thus the needles 44 protrude in a direction intersecting with the center axis Ax at an angle other than the right angle may be employed.

The first and second embodiments described above employ the configuration in which high frequency energy is applied from the needles 44 to a subject region; however, the configuration is not limited thereto and a configuration in which thermal energy is applied from the needles 44 to a subject region may be employed. Applying thermal energy to a subject region means transmission of heat from a heater, or the like, to the subject region.

In the first and second embodiments described above, the positional relationship between the first needles 44a and the second needles 44b is not limited to the positional relationship illustrated in FIG. 4 and another positional relationship may be employed.

According to the treatment tool according to the disclosure, it is possible to increase convenience.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
an insertion portion that is hollow and elongated, the insertion portion including a plurality of holes, each hole being configured to communicate between an inside and an outside of the insertion portion;
a shaft that is arranged in the insertion portion, the shaft being configured to move forward and backward in a longitudinal direction of the insertion portion;
a plurality of needles that are connected to the shaft, each needle being configured to move between a first position and a second position according to forward and backward move of the shaft, the first positon being a position in which the needle protrudes from the hole to the outside of the insertion portion, the second position being a positon in which the needle is positioned in the insertion portion;
a plurality of first insulating materials, each insulating material being configured to cover the needle; and
an energy supplier configured to supply electric power to the plurality of needles,
wherein the hole is configured to regulate a protruding direction of the needle to a direction intersecting with the longitudinal direction of the insertion portion, the protruding direction of the needle being a direction in which the needle protrudes to the outside of the insertion portion, and
when an area that is positioned outside the insertion portion out of a whole area of the needle at the first position is sectioned into two areas that are a first area and a second area, the first insulating material is configured to cover the second area, the first area being an area containing a distal end of the needle, the second area being an area excluding the first area.

2. The treatment tool according to claim 1, wherein the plurality of holes are configured to regulate protruding directions of the plurality of needles such that the protruding directions of the plurality of needles are parallel to each other, the protruding directions of the plurality of needles being directions in which the plurality of needles protrude to the outside of the insertion portion.

3. The treatment tool according to claim 1, wherein the first area is an energy emission area from which energy for treating living tissue is emitted according to the electric power that is supplied from the energy supplier.

4. The treatment tool according to claim 1, further comprising a plurality of second insulating materials, each second insulating material being configured to cover the needle,
wherein, when the first area is sectioned into two areas that are a third area and a fourth area, the second insulating material is configured to cover the third area, the third area being an area containing the distal end of the needle, the fourth area being an area excluding the third area, and
the fourth area is an energy emission area from which energy for treating living tissue is emitted according to the electric power that is supplied from the energy supplier.

5. The treatment tool according to claim 3, wherein all lengths of second areas of the plurality of needles are equal to one another, and
all lengths of energy emission areas of the plurality of needles are equal to one another.

6. The treatment tool according to claim 4, wherein all lengths of second areas of the plurality of needles are equal to one another, and
all lengths of energy emission areas of the plurality of needles are equal to one another.

7. The treatment tool according to claim 1, wherein each of the plurality of needles is formed of a superelastic wire.

8. The treatment tool according to claim 1, wherein the plurality of needles include
at least one first needle; and
at least one second needle that is set, by the energy supplier, at a potential different from a potential of the first needle, and
the energy supplier is configured to supply high frequency power between the at least one first needle and the at least one second needle.
